(19) United States
(12) Patent Application Publication
(10) Pub. No.: US 2010/0298329 A1
(43) Pub. Date: Nov. 25, 2010

(54) TOLPERISONE AND TOLPERISONE-LIKE DRUGS FOR THE TREATMENT OF K-RAS ASSOCIATED CANCERS

(75) Inventors: Alice T. Shaw, Chestnut Hill, MA (US); Tyler E. Jacks, West Newton, MA (US); Nicola Tolliday, Jamaica Plain, MA (US)

Correspondence Address: WOLF GREENFIELD & SACKS, P.C., 600 ATLANTIC AVENUE, BOSTON, MA, 02210-2206, US

(73) Assignee: Massachusette Institute of Technology, Cambridge, MA (US)

(21) Appl. No.: 12/678,495

(22) PCT Filed: Sep. 19, 2008
(86) PCT No.: PCT/US08/10909

§ 371 Date: Jun. 21, 2010

Related U.S. Application Data
(60) Provisional application No. 60/994,437, filed on Sep. 19, 2007.

(51) Int. Cl. A61K 31/4453; A61K 31/40; A61K 31/5375; C07D 211/32; C07D 265/30; A61P 35/00

(52) U.S. Cl. 514/238.8; 514/317; 514/428; 546/237; 544/175

(57) ABSTRACT

The invention provides compositions and methods for treating cancer. Aspects of the invention relate to therapeutic compositions comprising tolperisone and related compounds. Aspects of the invention relate to methods and compositions for treating Ras-associated cancers.

### RELATED APPLICATIONS

This application claims priority under 35 U.S.C. §119(e) to co-pending U.S. Provisional Application Ser. No. 60/994,437, filed Sep. 19, 2007, the contents of which are incorporated herein by reference.

### STATEMENT REGARDING FEDERALLY SPONSORED RESEARCH OR DEVELOPMENT

This invention was made with the support under the following government contracts: 20XS139A, awarded by the Science Applications International Corporation/National Cancer Institute, and 5-U01 CA84306-06, Project No. 6899457/6899456, awarded by the National Institute of Health/National Cancer Institute. The government has certain rights in the invention.

### FIELD OF THE INVENTION

The present invention generally relates to compositions and methods for treating subjects having Ras-associated conditions.

### BACKGROUND OF THE INVENTION

Activating mutations in the Ras oncogenes (H—, N—, and K-Ras) are found in approximately 30% of all human tumors. Certain tumor types can have a particularly high incidence of activating Ras mutations, including pancreatic, colon, and lung carcinomas. These mutations occur most frequently in K-Ras, and are predominantly missense mutations involving codons 12, 13, and 61. Mutations at these residues can compromise the GTPase activity of Ras, resulting in the accumulation of active, GTP-bound Ras, which may lead to the constitutive stimulation of Ras-responsive signaling pathways. Through the aberrant regulation of these signaling pathways, oncogenic Ras can mediate several aspects of malignant transformation, including deregulated cell growth, evasion of apoptosis, induction of angiogenesis, and propensity to metastasis. Thus, oncogenic activation of Ras contributes significantly to the development and progression of human neoplasia. Currently, Ras-targeted therapies for solid tumors have not exhibited sufficient antitumor activity to be clinically effective in treating patients with a Ras-associated malignancies. For example, farnesyltransferase inhibitors (FTIs) did not demonstrate antitumor activity in clinical trials of solid tumors.

Accordingly, improved methods and compositions are needed.

### SUMMARY OF THE INVENTION

The present invention provides methods and compositions for treating subjects who have Ras-associated cancers. In some aspects, one or more compositions of the invention are administered to a patient that is known to have a Ras-associated cancer. In some embodiments, a composition of the invention is prescribed and/or administered to a subject based on the subject having a Ras-associated cancer (e.g., prescribed and/or administered on the basis of a diagnosis of a Ras-associated cancer). For example, a subject may be selected for treatment with one or more compositions of the invention on the basis that the subject is known to have a Ras-associated cancer (e.g., on the basis of a positive diagnosis for a Ras-associated cancer). According to aspects of the invention, a Ras-associated cancer is a cancer that is associated with Ras activation. In some embodiments, Ras activation is caused by one or more mutations in the Ras gene. In certain embodiments, Ras activation is caused by Ras over-expression. In some embodiments, Ras activation results from the activation of one or more components of a Ras signaling pathway. It should be appreciated that as used herein, Ras activation refers to activation of the Ras gene, a Ras gene product (e.g., RNA and/or protein), or a combination thereof.

In some aspects, methods of the invention comprise administering a therapeutically effective amount of a compound having the following structure to a subject who has a Ras-associated cancer,

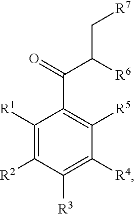

wherein each R¹⁻⁶ can be the same or different and is a hydrogen, halide, alkyl, heteroalkyl, alkenyl, heteroalkenyl, alkynyl, heteroalkynyl, aryl, heteroaryl, optionally substituted, and R⁷ is a heteroalkyl or heterocycle, optionally substituted, or a pharmaceutically acceptable salt thereof.

The present invention also relates to compositions of matter comprising a compound having the following structure,

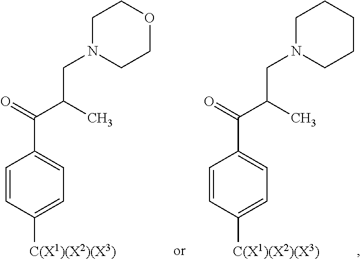

wherein each X¹⁻³ can be the same or different and is a hydrogen, halide, or alkyl.

The present invention also provides pharmaceutical compositions comprising a compound having the following structure,

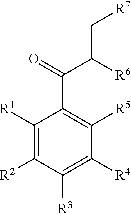

wherein each R¹⁻⁶ can be the same or different and is a hydrogen, halide, alkyl, heteroalkyl, alkenyl, heteroalkenyl, alkynyl, heteroalkynyl, aryl, heteroaryl, optionally substituted, and R⁷ is a heteroalkyl or heterocycle, optionally substituted, or a pharmaceutically acceptable salt thereof; and one or more pharmaceutically acceptable carriers, additives, and/or diluents.

In any of the above embodiments, each R¹⁻⁶ can be the same or different and may be a hydrogen, halide, alkyl, or aryl, optionally substituted. In any of the above embodiments, R¹, R², R⁴, and R⁵ may be hydrogen, and R⁶ may be methyl. In any of the above embodiments, R³ may be an alkyl substituted with one or more halides. In any of the above embodiments, R³ may be hydrogen, fluoro, methyl, ethyl, or trifluoromethyl. In any of the above embodiments, R⁷ may be a nitrogen heterocycle. In any of the above embodiments, R⁷ may be pyrrolidine, piperidine, or morpholine. In any of the above embodiments, the compound may have the following structure,

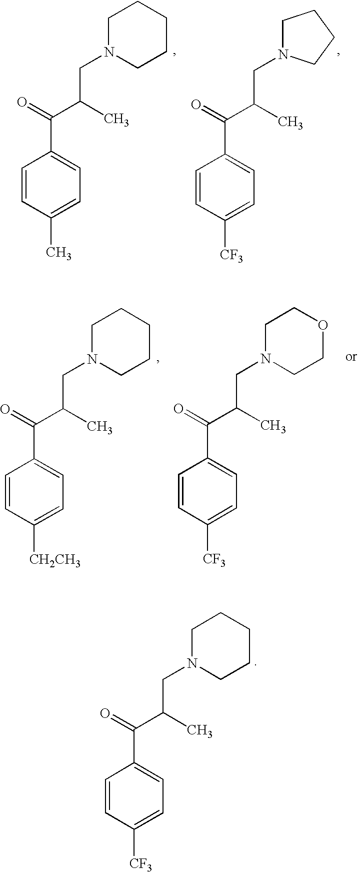

In any of the above embodiments, the subject may be a human. In any of the above embodiments, the Ras-associated cancer may be selected from the group consisting of: pancreatic cancer, colon cancer, and lung cancers. In any of the above embodiments, the compound may be administered orally, parenterally, subcutaneously, and/or intravenously. Any of the above embodiments can further comprise the act of determining that a subject has a Ras-associated cancer, prior to the act of prescribing and/or administering. For example, a subject may be selected for treatment with a composition of the invention on the basis that the subject is known to have a Ras-associated cancer (e.g., on the basis of a diagnosis of a Ras-associated cancer). The methods described herein can further comprise the act of monitoring the subject, after the act of administering, to determine a change in tumor size (e.g., tumor growth) or spread of tumor within a subject (e.g., to monitor or confirm that the administered composition and/or dosage is effective).

In any of the above embodiments, each X¹⁻³ may independently be any halide. In any of the above embodiments, the halide may be bromide, iodide, chloride, or fluoride.

In any of the above embodiments, the compound may have the following structure,

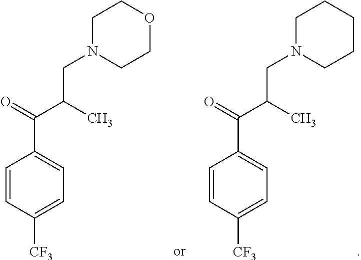

In any of the above embodiments, a pharmaceutical composition may comprise an enteric coating, a sustained release formulation or a lyophilized preparation. In any of the above embodiments, the pharmaceutical formulation may be a packaged unit dosage. In any of the above embodiments, the packaged unit dosage may be a solution. In some embodiments, when R³ is methyl or ethyl, R⁷ is not piperidine. In certain embodiments, when R³ is trifluoromethyl, R⁷ is not pyrrolidine. In some embodiments, when R³ is halide, R⁷ is not piperidine, pyrrolidine, homopiperidine, or a species having the structure,

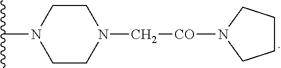

In certain embodiments, when R³ is hydrogen, R⁷ is not piperidine, pyrrolidine, homopiperidine, N-methyl piperazine, 4-methyl piperidine, N-cyclohexylamine, or N,N-dimethylamine.

The present invention also relates to the use of any of the compositions and/or compounds described herein in the preparation of a medicament for treating a subject having a Ras-associated cancer.

The present invention also provides methods for selectively inhibiting replication of cells which overexpress Ras and/or methods for inducing cell death in cells which overexpress Ras, relative to normal cells. The present invention also provides methods for selectively inhibiting replication of cells having one or more Ras mutations (e.g., Ras mutations associated with cancer). In some embodiments, the method comprises inducing oxidative stress in cells which overexpress Ras or in cells having one or more Ras mutations. In some embodiments, the method comprises inducing hypoxia in cells which overexpress Ras or in cells having one or more Ras mutations. In some embodiments, the method comprises producing species (e.g., oxygen species) which induce oxidative stress in cells which overexpress Ras or in cells having one or more Ras mutations. In some embodiments, the method comprises inhibiting the growth of cells characterized by the accumulation of active, GTP-bound Ras. In some embodiments, the method comprises inhibiting the growth of cells characterized by abnormally activated Ras-responsive signaling pathways. In some embodiments, the method comprises preventing the malignant transformation of normal cells into cells which overexpress Ras or cells which have one or more Ras mutations. In some embodiments, the method comprises inhibiting deregulated cell growth, evasion of apoptosis, angiogenesis, and/or metastasis associated with Ras mutations and/or Ras overexpression. The present invention also provides methods for decreasing the tumor size of a Ras-associated cancer.

Any of the above methods may comprise contacting a cell which overexpresses Ras with a compound of the invention. In certain embodiments, the contacting may occur via administration to the subject.

Accordingly, one or more compounds or compositions of the invention may be used to selectively inhibit replication of, or induce cell death of, cells which overexpress Ras or which have one or more Ras mutations, to induce oxidative stress or hypoxia in cells which overexpress Ras or which have one or more Ras mutations, to produce species which induce oxidative stress (e.g., oxygen species) in cells which overexpress Ras or which have one or more Ras mutations, to inhibit the growth of cells characterized by the accumulation of active, GTP-bound Ras and/or by abnormally activated Ras-responsive signaling pathways, to prevent the malignant transformation of normal cells into cells which overexpress Ras or cells which have one or more Ras mutations, to inhibit deregulated cell growth, evasion of apoptosis, angiogenesis, and/or metastasis associated with Ras mutations and/or Ras overexpression, or to decrease the tumor size of a Ras-associated cancer, in a subject.

In some embodiments, the method comprises treating a solid tumor (e.g., decreasing the size or preventing the growth of a solid tumor). In some embodiments, the method comprises treating a hematologic malignancy.

Methods of the invention comprise inhibiting or reducing any of the above symptoms or processes associated with cancer by about 1%, about 5%, about 10%, about 20%, about 30%, about 40%, about 50%, about 60%, about 70%, about 80%, about 90%, or, in some embodiments, 100%, relative to a non-treated cancer.

These and other aspects of the invention are described in more detail in the following detailed description.

### BRIEF DESCRIPTION OF THE DRAWINGS

FIG. 1 shows the synthesis of lanperisone, according to one embodiment of the invention.

FIG. 2 shows the activity of (a) tolperisone, (b) lanperisone, and (c) eperisone in inhibiting K-Ras mutant cells.

FIG. 3A shows tunnel staining data for (i) DMSO-treated wild type cells, (ii) lanperisone-treated wild type cells, (iii) DMSO-treated K-Ras mutant cells, and (iv) lanperisone-treated K-Ras mutant cells.

FIG. 3B shows cellular DNA content for (i) DMSO-treated wild type cells, (ii) lanperisone-treated wild type cells, (iii) DMSO-treated K-Ras mutant cells, and (iv) lanperisone-treated K-Ras mutant cells.

FIG. 3C shows BrdU staining data for (i) DMSO-treated wild type cells, (ii) lanperisone-treated wild type cells, (iii) DMSO-treated K-Ras mutant cells, and (iv) lanperisone-treated K-Ras mutant cells.

FIG. 4 shows graphs of (a) initial tumor volume, (b) final tumor volume, and (c) body weight for xenografts harboring subcutaneous K-ras G12D-expressing mouse fibroblast tumors, when treated with control DMSO or lanperisone.

Other aspects, embodiments and features of the invention will become apparent from the following detailed description when considered in conjunction with the accompanying drawings. The accompanying figures are schematic and are not intended to be drawn to scale. For purposes of clarity, not every component is labeled in every figure, nor is every component of each embodiment of the invention shown where illustration is not necessary to allow those of ordinary skill in the art to understand the invention. All patent applications and patents incorporated herein by reference are incorporated by reference in their entirety. In case of conflict, the present specification, including definitions, will control.

### DETAILED DESCRIPTION

The invention provides methods and compositions useful for treating subjects having cancer or at risk of developing cancer. In some embodiments, methods and compositions of the invention are useful for treating Ras-associated cancers. In some aspects, the invention provides compounds and related compositions for use in treating subjects diagnosed with cancer or at risk of developing cancer. In some aspects, the invention provides compounds and related compositions for treating a subject known to have (or be at risk of developing) a Ras-associated cancer. In some embodiments, a method of the invention includes administering to a subject a therapeutically effective amount of a compound or a therapeutic preparation, composition, or formulation of the compound such as those described herein, including those in the Summary, Claims, Figures, and patents and publications listed herein. In preferred embodiments, the subject is a human.

Aspects of the invention are based, at least in part, on the recognition that certain compounds are preferentially effective against Ras-associated cancers. In some embodiments, the invention includes administering a therapeutically effective amount of one or more of the following compounds to a subject that has cancer. In some embodiments, the compound is administered only to subjects that are selected on the basis that they are known to have a Ras-associated cancer. In certain aspects, a compound having the following structure is effective against Ras-associated cancers:

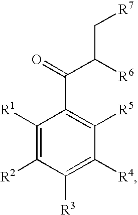

wherein each R¹⁻⁶ can be the same or different and is a hydrogen, halide, alkyl, heteroalkyl, alkenyl, heteroalkenyl, alkynyl, heteroalkynyl, aryl, heteroaryl, optionally substituted, and R⁷ is heteroalkyl or heterocycle, optionally substituted, or a pharmaceutically acceptable salt thereof. In some cases, each R¹⁻⁶ can be the same or different and is a hydrogen, halide, alkyl, or aryl, optionally substituted.

In some embodiments, R⁶ is methyl. In some embodiments, R¹, R², R⁴, and R⁵ are hydrogen, and R⁶ is methyl.

In some embodiments, R³ may be hydrogen, halide, such as fluoro, chloro, bromo, or iodo, methyl, ethyl, propyl, or other alkyl groups. R³ may also be an alkyl group substituted with one or more halides. For example, R³ may be a monohalomethyl, a dihalomethyl, or trihalomethyl groups. In some embodiments, R³ may be a trifluoromethyl group.

In some embodiments, R⁷ may be a nitrogen heterocycle, such as pyrrolidine, piperidine, or morpholine. R⁷ may also be other heterocycle groups, as described more fully below. In other embodiments, R⁷ may be any cyclic structure, including aromatic and non-aromatic carbocyclic structures, such as phenyl or cyclohexyl, for example.

In some embodiments, when R³ is methyl or ethyl, R⁷ is not piperidine. In some embodiments, when R³ is trifluoromethyl, R⁷ is not pyrrolidine. In some embodiments, when R³ is halide, R⁷ is not piperidine, pyrrolidine, homopiperidine, or a species having the structure,

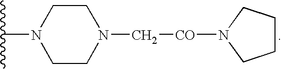

In some embodiments, when R³ is hydrogen, R⁷ is not piperidine, pyrrolidine, homopiperidine, N-methyl piperazine, 4-methyl piperidine, N-cyclohexylamine, or N,N-dimethylamine.

In some embodiments, R⁷ may be a non-cyclic structure such as a substituted amine (e.g., N,N-dialkyl amines such as N,N-dimethylamine, N-monoalkylamines such as N-cyclohexyl amine, and the like).

In some embodiments, the compound has the following structure,

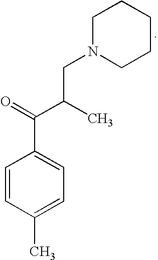

In some embodiments, the compound has the following structure,

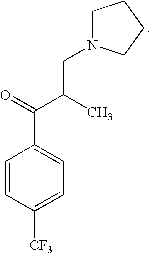

In some embodiments, the compound has the following structure,

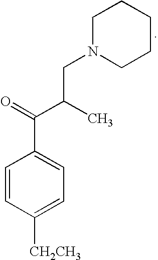

In some embodiments, the compound has the following structure,

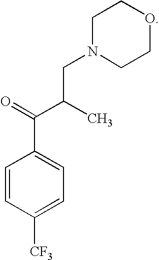

In some embodiments, the compound has the following structure,

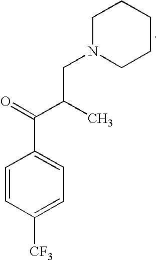

The compound may be orally administered, parenterally administered, subcutaneously administered, and/or intravenously administered.

A method of the invention may further comprise the act of determining that a subject has a Ras-associated cancer, or is at risk of developing a Ras-associated cancer, prior to prescribing and/or administering a composition described herein. In some embodiments, subjects are selected on the basis that they are known (e.g., diagnosed) to have a Ras-associated cancer, and a composition of the invention is prescribed and/or administered to the selected subjects on the basis of their known Ras-associated cancer.

In some embodiments, the invention also provides compositions of matter comprising a compound having the following structure,

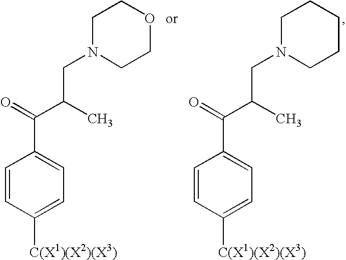

wherein each X¹⁻³ can be the same or different and is hydrogen, halide, or alkyl. In some cases, at least one of X¹⁻³ is a halide. In some cases, each X¹⁻³ is a halide. The halide may be bromide, iodide, chloride, or fluoride.

In some embodiments, the compound has the following structure,

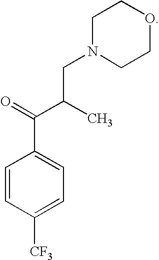

In some embodiments, the compound has the following structure,

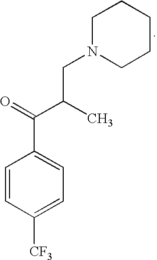

The present invention also provides pharmaceutical compositions comprising at least one of the compounds described herein, and one or more pharmaceutically acceptable carriers, additives, and/or diluents. For example, the pharmaceutical composition may comprise an enteric coating, a sustained release formulation or a lyophilized preparation. In some cases, the pharmaceutical formulation is a packaged unit dosage. The packaged unit dosage may be a solution.

In some embodiments, the composition may comprise at least one of the compounds described herein in an amount that is at least 0.01 wt %, at least 0.1 wt %, at least 1 wt %, or, in some cases, at least 10 wt %, of the composition. In some embodiments, the composition may comprise at least one of the compounds described herein in an amount that is between 0.01 wt % and 10 wt %, between 0.01 wt % and 0.1 wt %, or, in some cases, between 0.1 wt % and 1 wt %, of the composition. However, it should be understood that the compound may be present in higher, lower, or intermediate amounts, and the invention is not limited in this respect. In some cases, the wt % may be weight per weight % of the composition. In some cases, the wt % may be weight per volume of the composition.

It should be appreciated that in any of the aspects or embodiments described herein, the compound(s) may be provided in any suitable stereoisomeric form, and/or pharmaceutically acceptable acid or base addition salt form, and in a therapeutically effective amount.

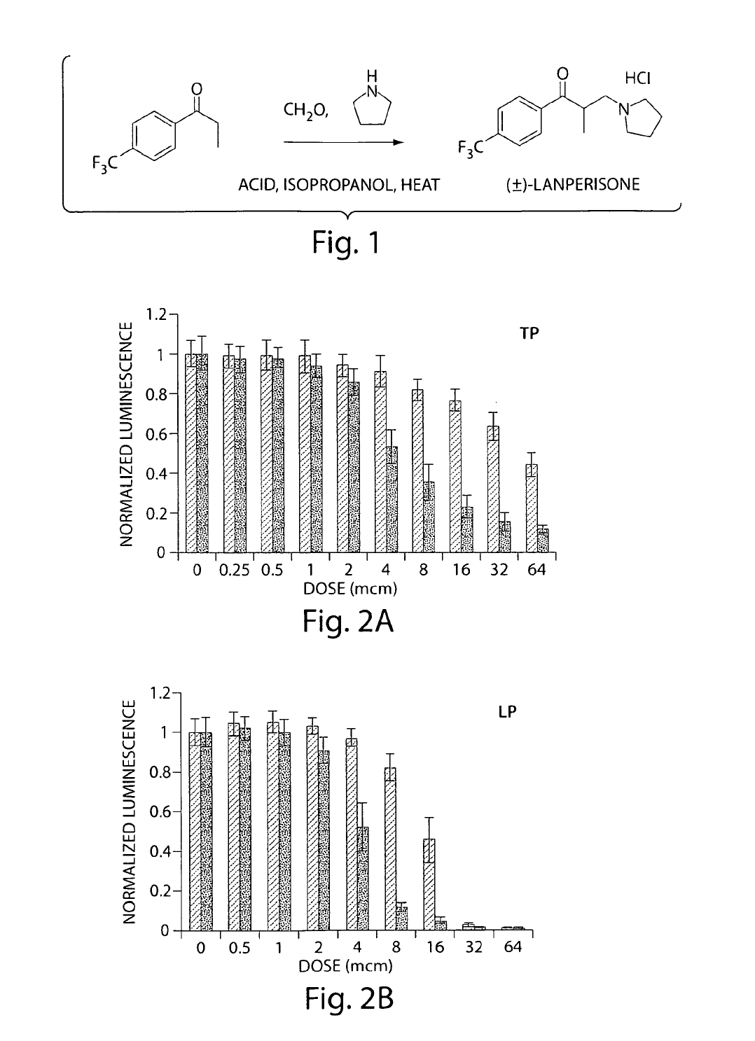

The compounds described herein may be synthesized using methods known in the art. For example, in an illustrative embodiment, FIG. 1 shows the synthesis of lanperisone by reaction (e.g., condensation) of a substituted ketone with formaldehyde and pyrrolidine, in the presence of acid. Those of ordinary skill in the art would be able to modify known methods such as this to synthesize the compounds as described herein. For example, the method shown in FIG. 1 may be modified to react a substituted ketone with formaldehyde and a heterocycle to form lanperisone, tolperisone, eperisone, and/or substituted derivatives thereof, based on the selection of the substituted ketone and/or heterocycle.

Aspects of the invention relate to administering one or more compounds or compositions of the invention to a subject having cancer, and particularly to a subject having a Ras-associated cancer. According to certain aspects of the invention, a Ras-associated cancer is a cancer that is associated with Ras activation. In some embodiments, Ras activation is caused by one or more mutations in the Ras gene. In certain embodiments, Ras activation is caused by Ras over-expression. Ras over-expression may be evaluated by comparing the level of Ras expression of in Ras-associated cancer cells with that of normal cells of similar tissue type. In some embodiments, Ras activation results from the activation of one or more components of a Ras signaling pathway. However, it should be appreciated that in some embodiments, a Ras-associated cancer may be a cancer caused by activation of a downstream component (e.g., regulatory protein) in a Ras signaling pathway without Ras itself being activated.

A cancer may be identified as a Ras-associated cancer using various methods, such as methods involving resection or biopsy of a tumor. In some cases, a biological fluid (e.g., blood) may be evaluated in order to identify whether a Ras-associated cancer is present within a sample or within a subject.

In some embodiments, the cancer may be a solid tumor. In some embodiments, the cancer may be a hematologic malignancy.

Accordingly, in certain embodiments, a mutation may be a mutation in a coding region of a Ras gene (e.g., K-Ras, H-Ras, and/or N-Ras) that alters the amino acid length and/or sequence of the encoded protein. For example, a Ras-associated cancer may be a cancer associated with a missense mutation in one or more of K-Ras codons 12, 13, and 16. However, one or more mutations (e.g., missense or nonsense mutations) in other codons of the K-Ras gene also may result in a Ras-associated cancer. In some embodiments, subjects having a mutation at one or more of K-Ras codons 14, 58, and 156 are predisposed to cancer and may be identified or selected for treatment with one or more compositions of the invention.

In some embodiments, a Ras-associated cancer may be associated with an activating Raf mutation, an NF-1 loss of function mutation, an activating Mek mutation, or any other mutation leading to constitutive activation of a Ras signaling pathway (e.g., in any gene encoding a regulatory component of the Ras signaling pathway), or any combination of two or more thereof.

In some embodiments, a mutation may be in a regulatory region (e.g., promoter, upstream or downstream regulatory sequence, etc.) that increases or decreases gene expression levels of one or more genes in the Ras signaling pathway (e.g., that increases Ras expression, etc.).

A cancer may be a cancer of any tissue or organ, including but not limited to one or more of the following: mouth, throat, lung, stomach, liver, pancreas, colon, rectum, bladder, breast, prostate, ovary, thyroid, etc., or any combination thereof. Accordingly, a Ras-associated cancer may in any tissue or organ in which tumors are associated with K-Ras activation (e.g., with a mutation in the K-Ras gene). For example, a cancer associated with K-Ras activation may be associated with a pancreatic tumor, a lung tumor (e.g., a non-small cell adenocarcinoma), a colorectal tumor, a thyroid tumor (e.g., a follicular or papillary thyroid tumor such as an undifferentiated papillary thyroid tumor), a seminoma, or myelodysplastic syndrome. In some embodiments, a Ras-associated cancer may in any tissue or organ in which tumors are associated with H-Ras activation (e.g., with a mutation in the H-Ras gene). For example, a cancer associated with H-Ras activation may be associated with a thyroid tumor (e.g., a follicular or papillary thyroid tumor such as an undifferentiated papillary thyroid tumor), a bladder tumor, or a kidney tumor. In certain embodiments, a Ras-associated cancer may in any tissue or organ in which tumors are associated with N-Ras activation (e.g., with a mutation in the N-Ras gene). For example, a cancer associated with N-Ras activation may be associated with a thyroid tumor (e.g., a follicular or papillary thyroid tumor such as an undifferentiated papillary thyroid tumor), a seminoma, a melanoma, a liver tumor, myelodysplastic syndrome, or acute myelogenous leukemia. In some embodiments, compositions and methods of the invention may be used to treat one or more hematologic malignancies that harbor Ras mutations. In some embodiments, a Ras-associated cancer may be in any tissue or organ in which tumors are associated with a mutation in the BRAF gene (e.g., melanomas or colorectal tumors). In certain embodiments, a Ras-associated cancer may be in any tissue or organ in which tumors are associated with EGFR over-expression (e.g., most carcinomas). In some embodiments, a Ras-associated cancer may be in any tissue or organ in which tumors are associated with ERBB2 over-expression (e.g., breast tumors). In certain embodiments, a Ras-associated cancer may be in any tissue or organ in which tumors are associated with loss of the PTEN gene (e.g., in glioblastoma multiforme, prostate tumors, or pancreatic tumors). In some embodiments, a Ras-associated cancer may be in any tissue or organ in which tumors are associated with AKT2 amplification (e.g., ovarian or pancreatic tumors). In certain embodiments, a Ras-associated cancer may be in any tissue or organ in which tumors are associated with PI3K amplification (e.g., ovarian tumors).

A subject may be human. A subject may be a subject diagnosed with cancer or otherwise known to have cancer. In some embodiments, a subject may be diagnosed as, or known to be, at risk of developing cancer. In some embodiments, a subject may be diagnosed with, or otherwise known to have, a Ras-associated cancer. In certain embodiments, a subject may be selected for treatment on the basis of a known Ras-associated cancer in the subject. In some embodiments, a subject may be selected for treatment on the basis of a suspected Ras-associated cancer in the subject. A Ras-associated cancer may be diagnosed by detecting a Ras mutation in a biopsy of a cancerous tissue or organ. In some embodiments, a Ras-associated cancer may be diagnosed by detecting a Ras mutation in a biological sample (e.g., urine, sputum, whole blood, serum, stool, etc., or any combination thereof. Accordingly, a compound or composition of the invention may be administered to a subject based, at least in part, on the fact that a Ras mutation is detected in at least one sample (e.g., biopsy sample or any other biological sample) obtained from the subject. In some embodiments, a cancer may not have been detected or located in the subject, but the presence of a Ras mutation in at least one biological sample may be sufficient to prescribe or administer one or more compositions of the invention to the subject. In some embodiments, the composition may be administered to prevent the development of a Ras-associated cancer. However, in some embodiments, the presence of an existing Ras-associated cancer may be suspected, but not yet identified, and a composition of the invention may be administered to prevent further growth or development of the cancer.

It should be appreciated that any suitable technique may be used to identify or detect a Ras mutation and/or Ras over-expression. For example, nucleic acid detection techniques (e.g., sequencing, hybridization, etc.) or peptide detection techniques (e.g., sequencing, antibody-based detection, etc.) may be used. In some embodiments, other techniques may be used to detect or infer the presence of a Ras-associated cancer (e.g., histology, etc.). For example, in lung cancer the histology of a tumor (e.g., the mucinous subtype) can be used to infer the presence of a K-Ras mutation.

In other aspects, the presence of a Ras-associated cancer can be detected or inferred by detecting a mutation, over-expression, amplification, or any combination thereof at one or more other loci associated with a Ras signaling pathway as described herein.

Aspects of the invention may be used to prevent the growth of a tumor or cancer, and/or to prevent the metastasis of a tumor or cancer. In some embodiments, compositions of the invention may be used to shrink or destroy a cancer. It should be appreciated that compositions of the invention may be used alone or in combination with one or more additional anti-cancer agents or treatments (e.g., chemotherapeutic agents, targeted therapeutic agents, pseudo-targeted therapeutic agents, hormones, radiation, surgery, etc., or any combination of two or more thereof). In some embodiments, one or more farnesyl transferase inhibitors may be used in combination with one or more compounds of the invention. A farnesyl transferase inhibitor may be considered a pseudo-targeted therapeutic agent in that it specifically inhibits farnesylation of not only Ras, but also several additional farnesylation targets. In some embodiments, a composition of the invention may be administered to a patient who has undergone a treatment involving surgery, and/or radiation, and/or chemotherapy. In certain embodiments, a composition of the invention may be administered chronically to prevent, or reduce the risk of, a cancer recurrence (particularly recurrence of a Ras-associated cancer).

The phrase “therapeutically-effective amount” as used herein means that amount of a compound, material, or composition comprising a compound of the present invention which is effective for producing some desired therapeutic effect in a subject at a reasonable benefit/risk ratio applicable to any medical treatment. Accordingly, a therapeutically effective amount prevents, minimizes, or reverses disease progression associated with a Ras-associated cancer. Disease progression can be monitored by clinical observations, laboratory and imaging investigations apparent to a person skilled in the art. A therapeutically effective amount can be an amount that is effective in a single dose or an amount that is effective as part of a multi-dose therapy, for example an amount that is administered in two or more doses or an amount that is administered chronically.

The effective amount of any one or more compounds may be from about 10 ng/kg of body weight to about 1000 mg/kg of body weight, and the frequency of administration may range from once a day to once a month. However, other dosage amounts and frequencies also may be used as the invention is not limited in this respect. A subject may be administered one or more compounds described herein in an amount effective to treat one or more cancers described herein. In one embodiment, the compound(s) may be one or more of the following: tolperisone, lanperisone, eperisone, or substituted derivatives thereof.

In the compounds and compositions of the invention, the term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. In some embodiments, a straight chain or branched chain alkyl may have 30 or fewer carbon atoms in its backbone, and, in some cases, 20 or fewer. In preferred embodiments, a straight chain or branched chain alkyl has 12 or fewer carbon atoms in its backbone (e.g., C₁-C₁₂ for straight chain, C₃-C₁₂ for branched chain), and more preferably 6 or fewer, and even more preferably 4 or fewer. Likewise, preferred cycloalkyls have from 3-10 carbon atoms in their ring structure, and more preferably have 5, 6 or 7 carbons in the ring structure. Examples of alkyl groups include, but are not limited to, methyl, ethyl, propyl, isopropyl, cyclopropyl, butyl, isobutyl, tert-butyl, cyclobutyl, hexyl, cyclochexyl, and the like.

The term “heteroalkyl” refers to an alkyl group as described herein in which one or more carbon atoms is replaced by a heteroatom. Suitable heteroatoms include oxygen, sulfur, nitrogen, phosphorus, and the like. Examples of heteroalkyl groups include, but are not limited to, alkoxy, amino, thioester, and the like.

The terms “alkenyl” and “alkynyl” refer to unsaturated aliphatic groups analogous in length and possible substitution to the alkyls described above, but that contain at least one double or triple bond respectively.

The terms “heteroalkenyl” and “heteroalkynyl” refer to unsaturated aliphatic groups analogous in length and possible substitution to the heteroalkyls described above, but that contain at least one double or triple bond respectively.

Unless the number of carbons is otherwise specified, “lower alkyl” as used herein means an alkyl group, as defined above, but having from one to ten carbons, more preferably from one to six carbon atoms in its backbone structure, and even more preferably from one to four carbon atoms in its backbone structure. Likewise, “lower alkenyl” and “lower alkynyl” have similar chain lengths. Preferred alkyl groups are lower alkyls. In preferred embodiments, a substituent designated herein as alkyl is a lower alkyl.

As used herein, the term “halogen” or “halide” designates —F, —Cl, —Br or —I.

The term “methyl” refers to the monovalent radical —CH₃, and the term “methoxyl” refers to the monovalent radical —CH₂OH.

The term “aralkyl” or “arylalkyl”, as used herein, refers to an alkyl group substituted with an aryl group.

The term “aryl” refers to aromatic carbocyclic groups, optionally substituted, having a single ring (e.g., phenyl), multiple rings (e.g., biphenyl), or multiple fused rings in which at least one is aromatic (e.g., 1,2,3,4-tetrahydronaphthyl, naphthyl, anthryl, or phenanthryl). That is, at least one ring may have a conjugated pi electron system, while other, adjoining rings can be cycloalkyls, cycloalkenyls, cycloalkynyls, aryls and/or heterocyclyls. The aryl group may be optionally substituted, as described herein. “Carbocyclic aryl groups” refer to aryl groups wherein the ring atoms on the aromatic ring are carbon atoms. Carbocyclic aryl groups include monocyclic carbocyclic aryl groups and polycyclic or fused compounds (e.g., two or more adjacent ring atoms are common to two adjoining rings) such as naphthyl groups. In some cases, the

The terms “heteroaryl” refers to aryl groups comprising at least one heteroatom as a ring atom.

The term “heterocycle” refers to refer to cyclic groups containing at least one heteroatom as a ring atom, in some cases, 1 to 3 heteroatoms as ring atoms, with the remainder of the ring atoms being carbon atoms. Suitable heteroatoms include oxygen, sulfur, nitrogen, phosphorus, and the like. In some cases, the heterocycle may be 3- to 10-membered ring structures, more preferably 3- to 7-membered rings, whose ring structures include one to four heteroatoms. The term “heterocycle” may include heteroaryl groups, saturated heterocycles (e.g., cycloheteroalkyl) groups, or combinations thereof. The heterocycle may be a saturated molecule, or may comprise one or more double bonds. In some case, the heterocycle is a nitrogen heterocycle, wherein at least one ring comprises at least one nitrogen ring atom. The heterocycles may be fused to other rings to form a polycylic heterocycle. The heterocycle may also be fused to a spirocyclic group. In some cases, the heterocycle may be attached to a compound via a nitrogen or a carbon atom in the ring.

Heterocycles include, for example, thiophene, benzothiophene, thianthrene, furan, tetrahydrofuran, pyran, isobenzofuran, chromene, xanthene, phenoxathiin, pyrrole, dihydropyrrole, pyrrolidine, imidazole, pyrazole, pyrazine, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, triazole, tetrazole, oxazole, isoxazole, thiazole, isothiazole, phenanthridine, acridine, pyrimidine, phenanthroline, phenazine, phenarsazine, phenothiazine, furazan, phenoxazine, pyrrolidine, oxolane, thiolane, oxazole, oxazine, piperidine, homopiperidine (hexamnethyleneimine), piperazine (e.g., N-methyl piperazine), morpholine, lactones, lactams such as azetidinones and pyrrolidinones, sultams, sultones, other saturated and/or unsaturated derivatives thereof, and the like. The heterocyclic ring can be optionally substituted at one or more positions with such substituents as described herein. In some cases, the heterocycle may be bonded to a compound via a heteroatom ring atom. In some cases, the heterocycle may be bonded to a compound via a carbon ring atom. In some cases, the heterocycle is pyrrolidine, piperidine, or morpholine.

The terms “amine” and “amino” are art-recognized and refer to both unsubstituted and substituted amines, e.g., a moiety that can be represented by the general formula: N(R′)(R″)(R′″) wherein R′, R″, and R′″ each independently represent a group permitted by the rules of valence.

The terms “ortho”, “meta” and “para” apply to 1,2-, 1,3- and 1,4-disubstituted benzenes, respectively. For example, the names 1,2-dimethylbenzene and ortho-dimethylbenzene are synonymous.

As used herein, the definition of each expression, e.g. alkyl, m, n, etc., when it occurs more than once in any structure, is intended to be independent of its definition elsewhere in the same structure.

As used herein, the term “substituted” is contemplated to include all permissible substituents of organic compounds, “permissible” being in the context of the chemical rules of valence known to those of ordinary skill in the art. It will be understood that “substituted” also includes that the substitution results in a stable compound, e.g., which does not spontaneously undergo transformation such as by rearrangement, cyclization, elimination, etc. In some cases, “substituted” may generally refer to replacement of a hydrogen with a substituent as described herein. However, “substituted,” as used herein, does not encompass replacement and/or alteration of a key functional group by which a molecule is identified, e.g., such that the “substituted” functional group becomes, through substitution, a different functional group. For example, a “substituted aldehyde” must still comprise the aldehyde moiety and can not be modified by substitution, in this definition, to become, e.g., a carboxylic acid. In a broad aspect, the permissible substituents include acyclic and cyclic, branched and unbranched, carbocyclic and heterocyclic, aromatic and nonaromatic substituents of organic compounds. Illustrative substituents include, for example, those described herein. The permissible substituents can be one or more and the same or different for appropriate organic compounds. For purposes of this invention, the heteroatoms such as nitrogen may have hydrogen substituents and/or any permissible substituents of organic compounds described herein which satisfy the valencies of the heteroatoms.

Examples of substituents include, but are not limited to, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, aryl, aryloxy, perhaloalkoxy, aralkoxy, heteroaryl, heteroaryloxy, heteroarylalkyl, heteroaralkoxy, azido, amino, halide, alkylthio, oxo, acylalkyl, carboxy esters, -carboxamido, acyloxy, aminoalkyl, alkylaminoaryl, alkylaryl, alkylaminoalkyl, alkoxyaryl, arylamino, aralkylamino, alkylsulfonyl, -carboxamidoalkylaryl, -carboxamidoaryl, hydroxyalkyl, haloalkyl, alkylaminoalkylcarboxy-, aminocarboxamidoalkyl-, cyano, alkoxyalkyl, perhaloalkyl, arylalkyloxyalkyl, and the like.

The compounds that have acidic properties can be converted into their pharmaceutically acceptable base addition salts by treating the acid form with a suitable organic or inorganic base. Appropriate base salt forms include, for example, the ammonium salts, the alkali and earth alkaline metal salts, e.g. the lithium, sodium, potassium, magnesium, calcium salts and the like, salts with organic bases, e.g. the benzathine, N-methyl-D-glucamine, hydrabamine salts, and salts with amino acids such as, for example, arginine, lysine and the like.

The terms acid or base addition salt also comprise the hydrates and the solvent addition forms which the compounds are able to form. Examples of such forms are e.g. hydrates, alcoholates and the like.

The term stereochemically isomeric forms of compounds, as used herein, include all possible compounds made up of the same atoms bonded by the same sequence of bonds but having different three-dimensional structures which are not interchangeable, which the compounds may possess. Unless otherwise mentioned or indicated, the chemical designation of a compound encompasses the mixture of all possible stereochemically isomeric forms that the compound can take. The mixture can contain all diastereomers and/or enantiomers of the basic molecular structure of the compound. All stereochemically isomeric forms of the compounds both in pure form or in admixture with each other are intended to be embraced within the scope of the present invention.

Some of the compounds may also exist in their tautomeric forms. Such forms although not explicitly indicated in the above formula are intended to be included within the scope of the present invention.

The methods and structures described herein relating to compounds and compositions of the invention also apply to the pharmaceutically acceptable acid or base addition salts and all stereoisomeric forms of these compounds and compositions.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention. In certain embodiments, the present invention relates to a compound represented by any of the structures outlined herein, wherein the compound is a single stereoisomer.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Contemplated equivalents of the compounds described above include compounds which otherwise correspond thereto, and which have the same general properties thereof, wherein one or more simple variations of substituents are made which do not adversely affect the efficacy of the compound. In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants, which are in themselves known, but are not mentioned here.

For purposes of this invention, the chemical elements are identified in accordance with the Periodic Table of the Elements, CAS version, Handbook of Chemistry and Physics, 67th Ed., 1986-87, inside cover.

In another aspect, the present invention provides “pharmaceutically acceptable” compositions, which comprise a therapeutically effective amount of one or more of the compounds described herein, formulated together with one or more pharmaceutically acceptable carriers (additives) and/or diluents. As described in detail, the pharmaceutical compositions of the present invention may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream or foam; sublingually; ocularly; transdermally; or nasally, pulmonary and to other mucosal surfaces.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

As set out herein, certain embodiments of the present compounds may contain a basic functional group, such as amino or alkylamino, and are, thus, capable of forming pharmaceutically-acceptable salts with pharmaceutically-acceptable acids. The term “pharmaceutically-acceptable salts” in this respect refers to the relatively non-toxic, inorganic and organic acid addition salts of compounds of the present invention. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al. (1977) “Pharmaceutical Salts,” J. Pharm. Sci. 66:1-19.)

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, and the particular mode of administration. The amount of active ingredient that can be combined with a carrier material to produce a single dosage form will generally be that amount of the compound which produces a therapeutic effect. Generally, this amount will range from about 1% to about 99% of active ingredient, preferably from about 5% to about 70%, most preferably from about 10% to about 30%.

In certain embodiments, a formulation of the present invention comprises an excipient selected from the group consisting of cyclodextrins, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol, glycerol monostearate, and non-ionic surfactants; absorbents, such as kaolin and bentonite clay; lubricants, such as talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made in a suitable machine in which a mixture of the powdered compound is moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Dissolving or dispersing the compound in the proper medium can make such dosage forms. Absorption enhancers can also be used to increase the flux of the compound across the skin. Either providing a rate controlling membrane or dispersing the compound in a polymer matrix or gel can control the rate of such flux.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers, which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which in turn, may depend upon crystal size and crystalline form.

Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, which are compatible with body tissue.

In certain embodiments, a compound or pharmaceutical preparation is administered orally. In other embodiments, the compound or pharmaceutical preparation is administered intravenously. Alternative routs of administration include sublingual, intramuscular, and transdermal administrations.

When the compounds of the present invention are administered as pharmaceuticals, to humans and animals, they can be given per se or as a pharmaceutical composition containing, for example, 0.1% to 99.5% (more preferably, 0.5% to 90%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required to achieve the desired therapeutic effect and then gradually increasing the dosage until the desired effect is achieved.

In some embodiments, a compound or pharmaceutical composition of the invention is provided to a subject chronically. Chronic treatments include any form of repeated administration for an extended period of time, such as repeated administrations for one or more months, between a month and a year, one or more years, or longer. In many embodiments, a chronic treatment involves administering a compound or pharmaceutical composition of the invention repeatedly over the life of the subject. Preferred chronic treatments involve regular administrations, for example one or more times a day, one or more times a week, or one or more times a month. In general, a suitable dose such as a daily dose of a compound of the invention will be that amount of the compound that is the lowest dose effective to produce a therapeutic effect. Such an effective dose will generally depend upon the factors described above. Generally doses of the compounds of this invention for a patient, when used for the indicated effects, will range from about 0.0001 to about 100 mg per kg of body weight per day. Preferably the daily dosage will range from 0.001 to 50 mg of compound per kg of body weight, and even more preferably from 0.01 to 10 mg of compound per kg of body weight. However, lower or higher doses can be used. In some embodiments, the dose administered to a subject may be modified as the physiology of the subject changes due to age, disease progression, weight, or other factors.

If desired, the effective daily dose of the active compound may be administered as two, three, four, five, six or more sub-doses administered separately at appropriate intervals throughout the day, optionally, in unit dosage forms.

While it is possible for a compound of the present invention to be administered alone, it is preferable to administer the compound as a pharmaceutical formulation (composition) as described above.

The compounds according to the invention may be formulated for administration in any convenient way for use in human or veterinary medicine, by analogy with other pharmaceuticals.

In some embodiments, one or more compositions of the invention may be provided along with instructions (e.g., written instructions) for administration to a subject having a Ras-associated cancer. In certain embodiments, a kit may be provided containing at least one composition of the invention, along with instructions for treatment of a Ras-associated cancer and/or along with a further agent for treating a Ras-associated cancer (e.g., a farnesyl transferase inhibitor).

According to the invention, the term “treatment” includes prophylaxis and therapy, and includes managing a subject's symptoms of Ras-associated cancer and halting the progression of the Ras-associated cancer (e.g., cell replication, tumor growth, etc.). Treatment includes preventing or slowing the development of a Ras-associated cancer, and/or the onset of certain symptoms associated with a Ras-associated cancer in a subject with, or at risk of developing, a Ras-associated cancer or a related disorder. Therapy includes preventing or slowing the replication of cells which overexpress Ras of which have one or more Ras mutations in a subject with one or more compounds or compositions described herein. Therapy also includes decreasing the tumor size of a Ras-associated cancer or the amount of cells which overexpress Ras of which have one or more Ras mutations in a subject with one or more compounds or compositions described herein. It should be appreciated that the terms preventing and/or inhibiting may be used to refer to a partial prevention and/or inhibition (e.g., a percentage reduction, for example about 5%, about 10%, about 20%, about 30%, about 40%, about 50%, about 60%, about 70%, about 80%, about 90%, or higher or lower or intermediate percentages of reduction). However, in some embodiments, a prevention or inhibition may be complete (e.g., a 100% reduction or about a 100% reduction based on an assay).

In some embodiments, the effective amount is tissue specific.

Having now described some illustrative embodiments of the invention, it should be apparent to those skilled in the art that the foregoing is merely illustrative and not limiting, having been presented by way of example only. Numerous modifications and other illustrative embodiments are within the scope of one of ordinary skill in the art and are contemplated as falling within the scope of the invention. In particular, although many of the examples presented herein involve specific combinations of method acts or system elements, it should be understood that those acts and those elements may be combined in other ways to accomplish the same objectives. Acts, elements and features discussed only in connection with one embodiment are not intended to be excluded from a similar role in other embodiments. Further, for the one or more means-plus-function limitations recited in the following claims, the means are not intended to be limited to the means disclosed herein for performing the recited function, but are intended to cover in scope any means, known now or later developed, for performing the recited function. Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

### Examples

### Example 1

A high-throughput, chemical genetic screen was performed for small molecules that selectively inhibited the viability of mouse embryonic fibroblasts (MEFs) expressing oncogenic K-Ras compared to wild-type control MEFs. These MEFs have been extensively characterized at the molecular and cellular level. Both wild-type and mutant K-Ras MEFs were screened to allow identification of only those compounds which selectively inhibit the mutant line. The inclusion of wild-type cells allowed for the elimination of general cytotoxic compounds and for the targeting of molecular pathways activated by oncogenic K-Ras. To measure inhibition of cell growth, a screen was performed using Promega's Cell Titer Glo Assay, which measured cell viability based on intracellular levels of ATP, an indicator of metabolically active, and hence, viable, cells.

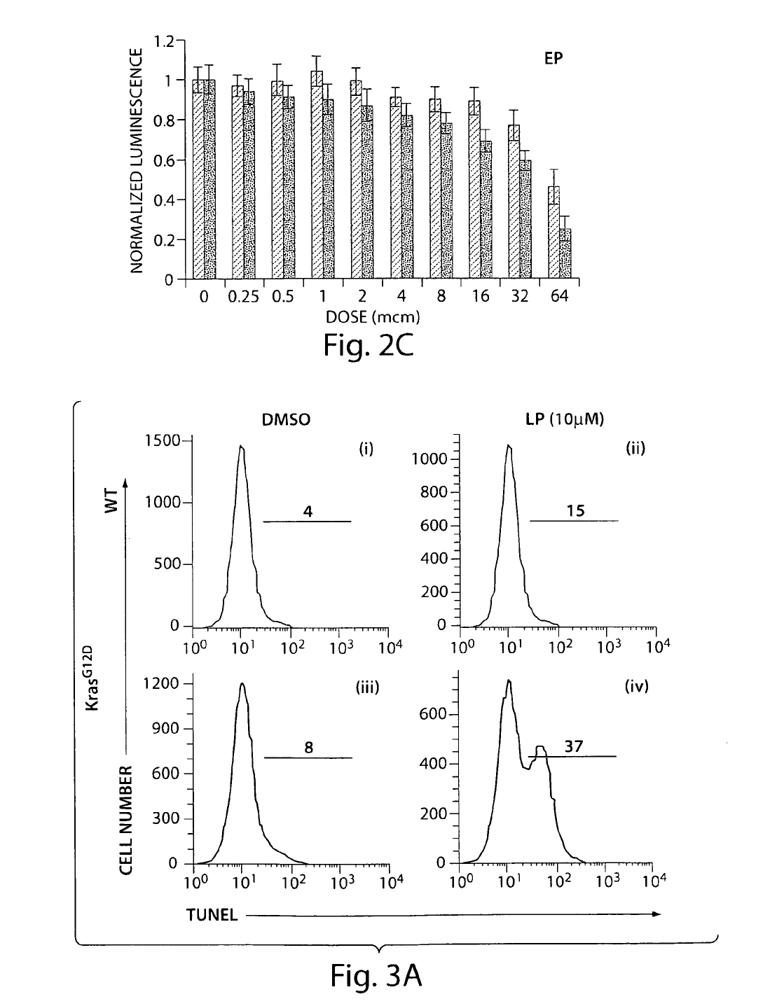

Among the >50,000 compounds screened, tolperisone was one compound which showed differential activity at concentrations as low as 5 microns. The differential activity of tolperisone confirmed using other assays, such as BrdU cytoblot assay, which measures proliferation based upon incorporation of BrdU. FIG. 2 shows the activity of (a) tolperisone, (b) lanperisone, and (c) eperisone in inhibiting K-Ras mutant cells. Structure-function analyses indicated that lanperisone also exhibits selectivity in inhibiting K-Ras mutant cells, with a selectivity of ˜10_{—}1 over wild-type cells.

### Example 2

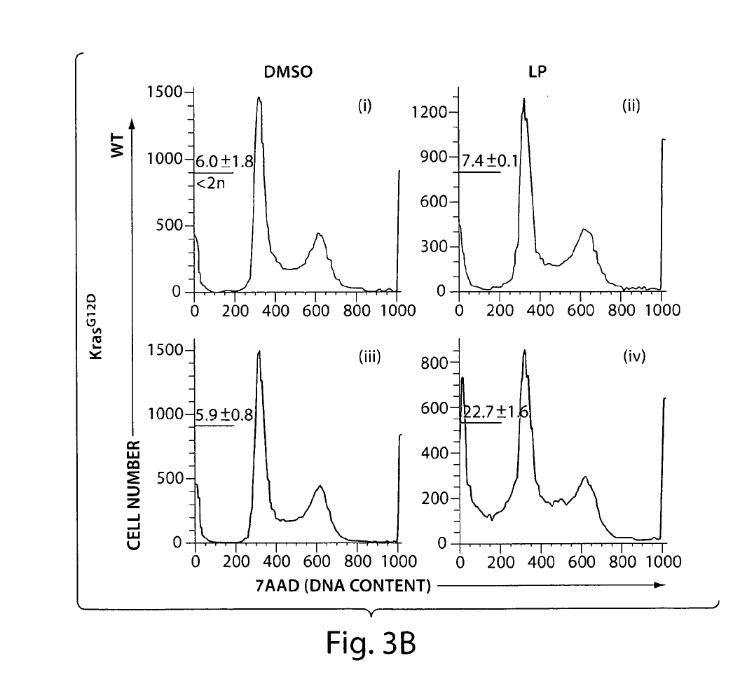

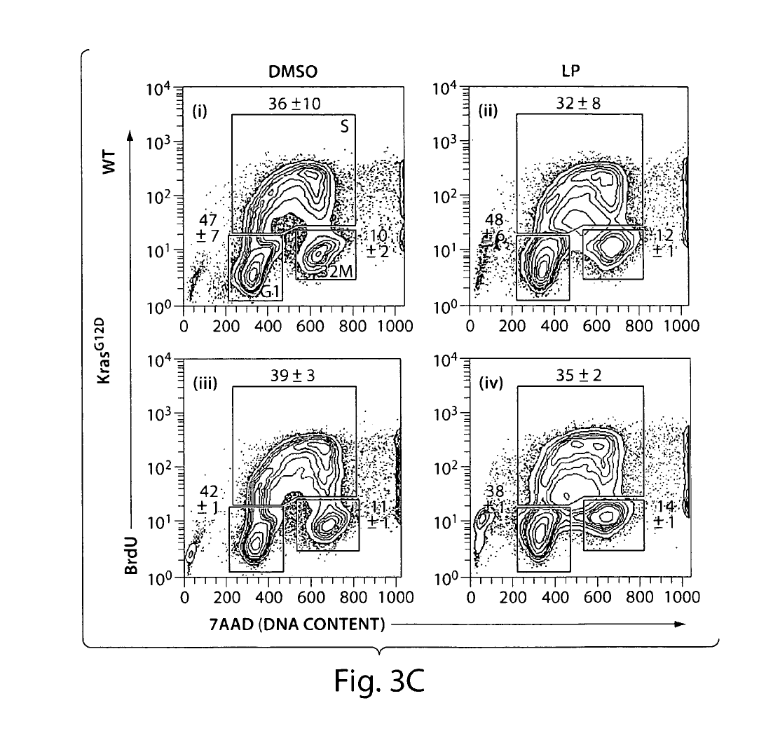

The effects of lanperisone on cell proliferation and cell death were evaluated based on a FACS analysis. Tunnel staining (for dying cells) and PI staining (excluded by living cells) were used to evaluate cell death in lanperisone and DMSO treated wild-type and K-Ras mutant cells (cells with a Kras G12D mutation). FIG. 3A shows tunel staining data for (i) DMSO-treated wild type cells, (ii) lanperisone-treated wild type cells, (iii) DMSO-treated K-Ras mutant cells, and (iv) lanperisone-treated K-Ras mutant cells. FIG. 3B shows cellular DNA content for (i) DMSO-treated wild type cells, (ii) lanperisone-treated wild type cells, (iii) DMSO-treated K-Ras mutant cells, and (iv) lanperisone-treated K-Ras mutant cells. FIG. 3C shows BrdU staining data for (i) DMSO-treated wild type cells, (ii) lanperisone-treated wild type cells, (iii) DMSO-treated K-Ras mutant cells, and (iv) lanperisone-treated K-Ras mutant cells.

The results indicated that, at 6 hours, lanperisone (at 10 μM and at 20 μM) preferentially caused cell death in K-Ras mutant cells (cells with Kras G12D mutation) relative to wild-type cells (FIG. 3A). Analysis of cellular DNA content showed that the cell death effect was not specific to any particular phase of the cell cycle (cells were found to be dying uniformly in all phases of the cell cycle (FIG. 3B). In addition, BrdU staining showed no specific defect in cellular proliferation (FIG. 3C).

### Example 3

To study the selectivity of lanperisone for K-ras mutant cells, gene expression profiling experiments were carried out using wild-type and K-rasG12D MEFs treated with lanperisone or control DMSO. The gene expression signatures associated with lanperisone treatment were compared with those observed upon 1) manipulation of any of a number of defined biological pathways (i.e., Gene Set Enrichment Analysis or GSEA) and 2) treatment with small molecules with known targets including FDA-approved drugs (ie the Connectivity Map or CMAP). Numerous connectivities were observed between lanperisone and other classes of small molecules, including calcium modulators, HDAC inhibitors, and HIF activators. GSEA demonstrated marked enrichment of hypoxia as well as oxidative stress pathways in lanperisone-treated MEFs. Additional biochemical experiments showed that lanperisone closely phenocopies inducers of oxidative stress, and that lanperisone selectively kills K-rasG12D-expressing cells by inducing reactive oxygen species. This mechanism of cell death occurs in both mouse and human cells, as well as in both fibroblast and epithelial cell lines.

### Example 4

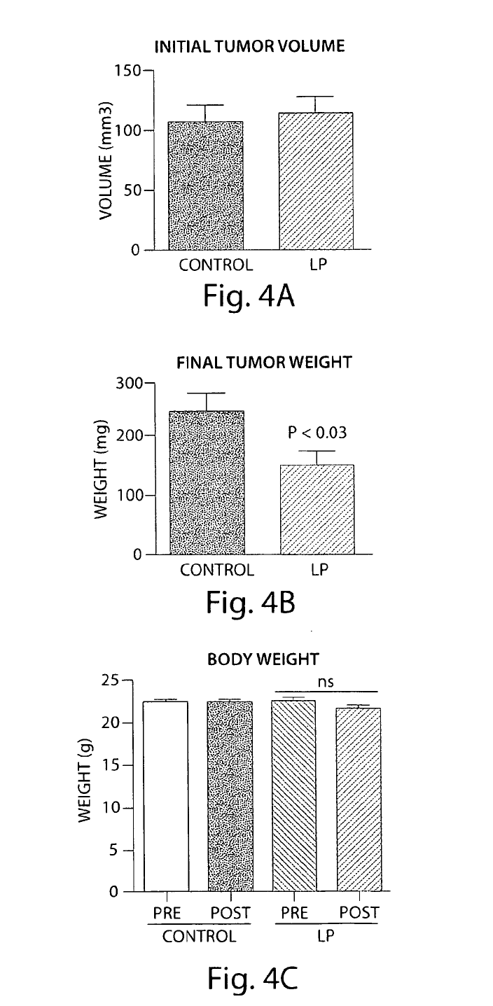

The effectiveness of one or more compounds of the invention (e.g., lanperisone) was evaluated in vivo using xenografts. The effect on xenograft tumor size was evaluated in mice by administering a compound of the invention at a dose of 100-200 mg/kg once a day (e.g., via oral administration in water). The compounds were also administered intraperitoneally. Mice harboring established, subcutaneous tumors measuring at least 1 cm were treated with lanperisone for 7 consecutive days. FIG. 4 shows graphs of (a) initial tumor volume, (b) final tumor volume, and (c) body weight for subcutaneous K-ras G12D-expressing mouse fibroblast tumors, when treated with control DMSO or lanperisone. At the end of the treatment period, treated animals showed significantly smaller tumors than matched controls. These findings confirm the in vivo efficacy of lanperisone in targeting K-ras mutant tumors.

What is claimed:

1. A method for treating a subject having cancer, the method comprising:
administering, to a subject selected on the basis that the subject is known to have a Ras-associated cancer, a therapeutically effective amount of a compound having the following structure, 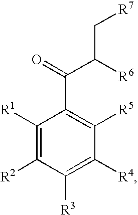
wherein each R¹⁻⁶ can be the same or different and is hydrogen, halide, alkyl, heteroalkyl, alkenyl, heteroalkenyl, alkynyl, heteroalkynyl, aryl, heteroaryl, optionally substituted, and
R⁷ is heteroalkyl or heterocycle, optionally substituted, or
a pharmaceutically acceptable salt thereof.

2. A method as in claim 1, wherein each R¹⁻⁶ can be the same or different and is hydrogen, halide, alkyl, or aryl, optionally substituted.

3. A method as in claim 1, wherein R¹, R², R⁴, and R⁵ are hydrogen, and R⁶ is methyl.

4. A method as in claim 1, wherein R³ is alkyl substituted with one or more halides.

5. A method as in claim 1, wherein R³ is hydrogen, fluoro, methyl, ethyl, or trifluoromethyl.

6. A method as in claim 1, wherein R⁷ is a nitrogen heterocycle.

7. A method as in claim 1, wherein R⁷ is pyrrolidine, piperidine, or morpholine.

8. A method as in claim 1, wherein the compound has the following structure,

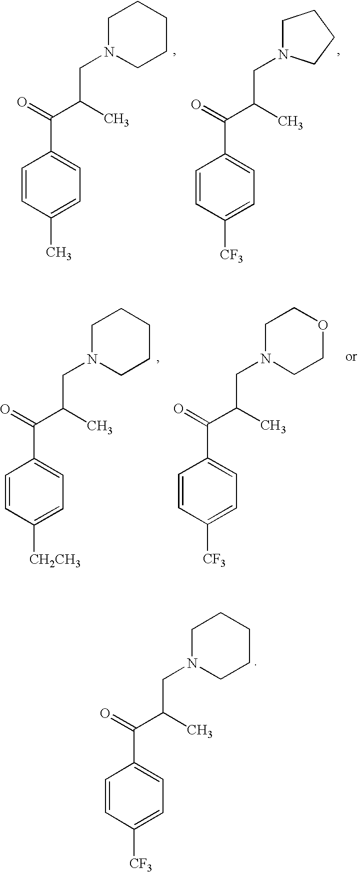

9. A method as in claim 1, wherein the subject is a human.

10. A method as in claim 1, wherein the Ras-associated cancer is selected from the group consisting of: pancreatic cancer, colon cancer, and lung cancer.

11. A method as in claim 1, wherein the compound is orally administered.

12. A method as in claim 1, wherein the compound is parenterally administered.

13. A method as in claim 1, wherein the compound is subcutaneously administered.

14. A method as in claim 1, wherein the compound is intravenously administered.

15. A method as in claim 1, further comprising the act of determining that the subject has a Ras-associated cancer, prior to the act of administering.

16. A method as in claim 1, further comprising the act of monitoring the subject, after the act of administering, to determine a change in tumor size.

17. A composition of matter, comprising:
a compound having the following structure, 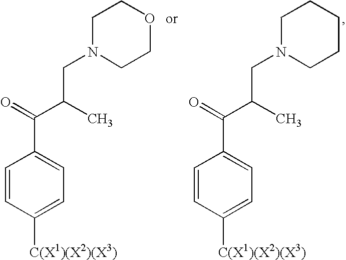
wherein each X¹⁻³ can be the same or different and is hydrogen, halide, or alkyl.

18. A composition as in claim 17, wherein each X¹⁻³ is a halide.

19. A composition as in claim 17, wherein the halide is bromide.

20. A composition as in claim 17, wherein the halide is iodide.

21. A composition as in claim 17, wherein the halide is chloride.

22. A composition as in claim 17, wherein the halide is fluoride.

23. A composition as in claim 17, wherein the compound has the following structure,

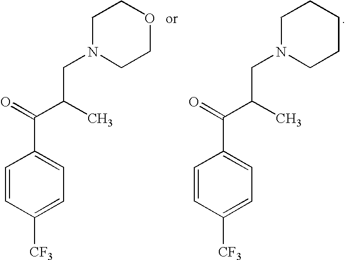

24. A pharmaceutical composition, comprising:
a compound having the following structure, 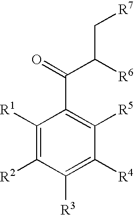
wherein each R¹⁻⁶ can be the same or different and is hydrogen, halide, alkyl, heteroalkyl, alkenyl, heteroalkenyl, alkynyl, heteroalkynyl, aryl, heteroaryl, optionally substituted, and
R⁷ is heteroalkyl or heterocycle, optionally substituted, or
a pharmaceutically acceptable salt thereof; and
one or more pharmaceutically acceptable carriers, additives, and/or diluents.

25. A pharmaceutical composition as in claim 24, wherein the pharmaceutical composition comprises an enteric coating, a sustained release formulation or a lyophilized preparation.

26. A pharmaceutical composition as in claim 24, wherein the pharmaceutical formulation is a packaged unit dosage.

27. A pharmaceutical composition as in claim 26, wherein the packaged unit dosage is a solution.

28. A pharmaceutical composition as in claim 24, wherein, when R³ is methyl or ethyl, R⁷ is not piperidine.

29. A pharmaceutical composition as in claim 24, wherein, when R³ is trifluoromethyl, R⁷ is not pyrrolidine.

30. A pharmaceutical composition as in claim 2, wherein, when R³ is halide, R⁷ is not piperidine, pyrrolidine, homopiperidine, or a species having the structure,

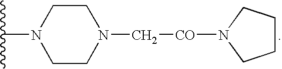

31. A pharmaceutical composition as in claim 24 wherein, when R³ is hydrogen, R⁷ is not piperidine, pyrrolidine, homopiperidine, N-methyl piperazine, 4-methyl piperidine, N-cyclohexylamine, or N,N-dimethylamine.
